(19) United States
(12) Patent Application Publication
(10) Pub. No.: US 2004/0254547 A1
(43) Pub. Date: Dec. 16, 2004

(54) Automatic urine disposal device and urine receptacle used therefor

(76) Inventors: Kenichi Okabe, Chiyoda (JP); Junichi Kobayashi, Ushiku (JP); Shigeru Machida, Iwama (JP); Ryousuke Miyagawa, Kasukabe (JP); Ichiro Wada, Toyohama (JP); Hiroaki Taniguchi, Toyohama (JP); Miou Suzuki, Toyohama (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/863,268

(22) Filed: Jun. 9, 2004

(30) Foreign Application Priority Data
Jun. 11, 2003 (JP) 2003-166671

(51) Int. Cl. A61M 1/00; A61F 13/15; A61F 13/20

(52) U.S. Cl. 604/317

(57) ABSTRACT

An automatic urine disposal device comprises a urine receptacle worn by a wearer and a vacuum pump for absorbing urine collected in the receptacle by means of a suction force. The urine receptacle is formed of a perforated urine absorbent sheet for absorbing urine discharged in a top sheet, and a support sheet in which a urine absorbing space forming sheet is interposed. The urine receptacle is connected to a sealed urine tank via a urine drainage tube. The vacuum pump absorbs air from the urine tank to draw urine from the urine receptacle to the urine tank.

### BACKGROUND OF THE INVENTION

The present invention relates to an automatic urine disposal device of the type worn by the bedridden elderly, hospitalized patients, physically disabled people, and others who are unable to voluntarily control their bladder or to clean up urine on their own, and it also relates to a urine receptacle used therefor.

Because of age, physical disability, hospitalization due to injury or illness, or other physical conditions, people sometimes become unable to voluntarily control their bladder or clean up urine on their own. In those situations, generally, a catheter is directly inserted into the bladder to discharge urine, or paper diaper is used.

When a catheter is directly inserted into the bladder, the wearer feels great discomfort, and there is also the probability of injuring the urethra or bladder or the occurrence of an infection. Thus, expertise as well as special sterilized utensils are required.

When a paper diaper is worn for a prolonged period of time, urine may leak, the wearer can become uncomfortable or get a stuffy feeling, or skin troubles, such as rashes, may occur. To avoid this, the paper diaper must be frequently changed, which will impose considerable physical and mental burdens on both the wearer and the caretaker. Imposed on a daily basis, those physical and mental burdens become a big concern and a significant economical burden as well.

To avoid those problems, a method has been presented in which urine that has been absorbed by a urine absorbent material in a urine receptacle is discharged by means of a vacuum pump and drawn into a urine tank. The vacuum pump sucks out air in a sealed urine tank, and, due to the difference between the tank's pressure and the atmospheric pressure, urine absorbed in the urine absorbent material is drained into the urine tank. Automatic urine disposal devices having such a configuration have been disclosed in Japanese Application Patent Laid-open Publication No. Hei 07-171182 and No. Hei 11-113946.

In the method disclosed in Japanese Application Patent Laid-open Publication No. Hei 07-171182 and No. Hei 11-113946, urine is drained from one location of the urine absorbent material, and, therefore, the percentage of urine collection from the urine receptacle (urine absorbent material) is low. For this reason, the amount of urine which remains in the urine receptacle (urine absorbent material) is large, which makes the wearer feel uncomfortable. To reduce the amount of urine which remains in the urine receptacle, the capacity of the vacuum pump must be increased. Accordingly, it becomes necessary to increase the size and volume of the urine disposal device.

### SUMMARY OF THE INVENTION

An object of the present invention is to provide a compact and lightweight automatic urine disposal device in which the percentage of urine collection by the urine receptacle is increased, and also, it is an object of the invention to provide a urine receptacle to be used therefor.

To achieve the aforementioned object, the present invention is designed such that it incorporates a urine receptacle in which a urine absorbing space forming sheet is disposed between a perforated urine absorbent sheet for absorbing urine discharged in a top sheet and a support sheet, and urine is drawn through a urine drainage port formed on the support sheet into a sealed urine tank by means of a vacuum pump via a urine drainage tube.

In other words, in accordance with the present invention, a urine absorbent layer is formed by using a urine absorbing space forming sheet disposed between a perforated urine absorbent sheet, on which a liquid-permeable, hard-breathable top sheet is located, and a non-breathable, liquid-impermeable support sheet, and urine is drained by making the pressure in a large number of urine drainage pores formed on the perforated urine absorbent sheet negative, so that urine is drawn into the urine tank via a urine drainage tube.

In the urine receptacle used in accordance with the present invention, urine which has been discharged in the top sheet is absorbed through a large number of urine drainage pores formed on the perforated urine absorbent sheet. Therefore, the percentage of urine collection from the urine receptacle increases and the amount of urine which remains in the urine receptacle is reduced. With the increase in the percentage of urine collection, a small-capacity vacuum pump with a low suction force can drain urine. Therefore, it is possible to drain urine from the urine receptacle without discomfort to the wearer, and the device can be compact and lightweight.

### BRIEF DESCRIPTION OF THE DRAWINGS

FIG. 1 is a diagram of an embodiment of the automatic urine disposal device of the present invention.

FIG. 2 through FIG. 4 are a top view, a bottom view, and an exploded perspective view of the urine receptacle, respectively.

FIG. 5 is an enlarged cross-sectional view taken along the line A-A in FIG. 2.

FIG. 6 is an enlarged plan view of a support sheet, which forms a component of the urine receptacle.

FIG. 7 is a diagram which illustrates the functions of the urine receptacle.

FIG. 8 is a diagram which shows another embodiment of a urine drainage tube.

### DETAILED DESCRIPTION OF THE INVENTION

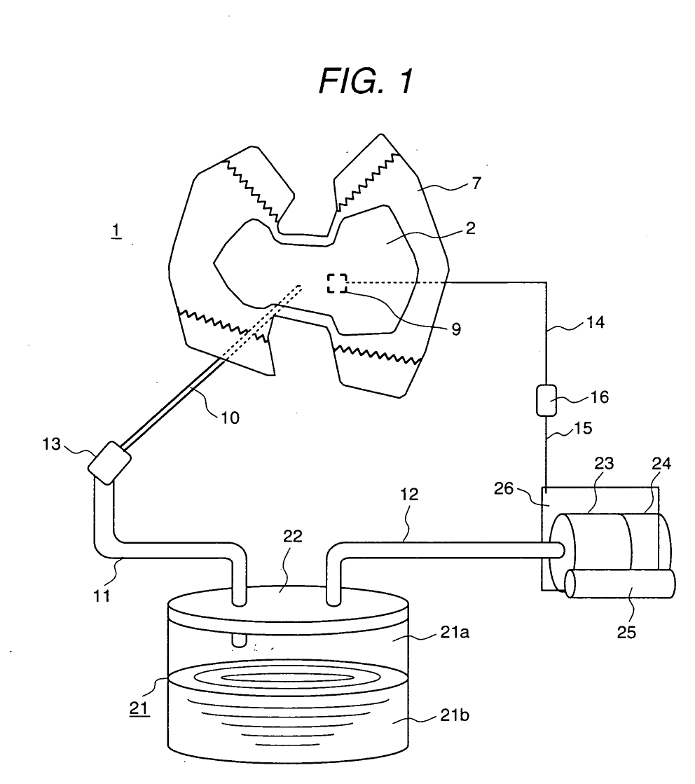

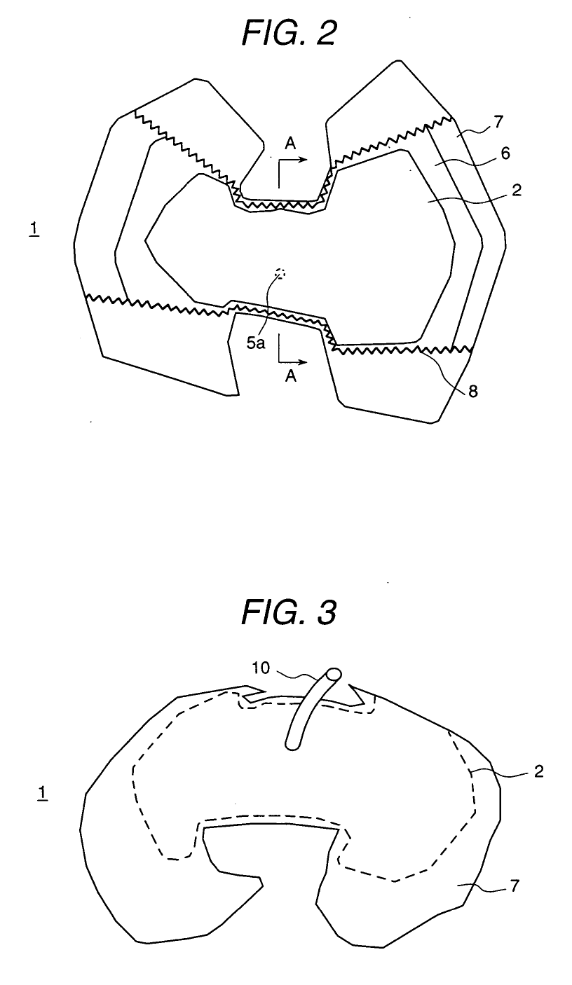

In the embodiment shown in FIGS. 1 through 6, a urine receptacle 1 which absorbs urine that has been discharged from a wearer's urinating part, not shown, is configured to have a concave shape, as shown in FIG. 3, and its width at the middle portion in the longitudinal direction (direction of the wearer's front and rear) 2 is narrow, so that it is shaped generally like an hourglass. The reason for this shape is to fit the wearer's crotch.

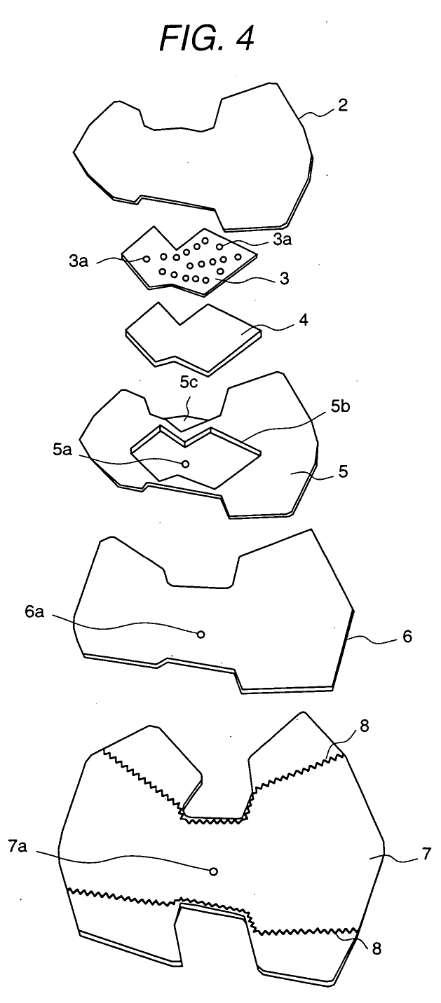

As shown in FIG. 4, the urine receptacle 1 consists of a top sheet 2, a perforated urine absorbent sheet 3, a urine absorbing space forming sheet 4, a support sheet 5, a urine absorbent sheet 6, an outer sheet 7 and gathers 8. The top sheet 2 is made of a liquid-permeable, hard-breathable nonwoven cloth, and the perforated urine absorbent sheet 3 is made of liquid-impermeable, non-breathable vinyl. The urine absorbing space forming sheet 4 is made of a rough fibrous material, which does not hold liquid, and the support sheet 5 is made of a liquid-impermeable, non-breathable, elastic polyethylene sheet, rubber, or vinyl. Furthermore, the urine absorbent sheet 6 is made of a compound material, which is formed of a liquid-permeable nonwoven cloth, flocculent pulp, and a polymer absorbent layer in combination, and the outer sheet 7 and gathers 8 are made of liquid-impermeable nonwoven cloths.

The outer sheet 7 is a thin, liquid-impermeable sheet, and it is made of a polyethylene film, for example. The outer sheet 7 may be simply made of a liquid-impermeable member; however, desirably, an optimal member can be chosen by taking into account stuffiness which may result from prolonged use. The outer surface of the outer sheet 7 is laminated with a soft and smooth surface material (not shown), such as a polypropylene nonwoven cloth, to prevent the wearer from becoming uncomfortable. The inner surface of the outer sheet 7 has been treated with a water-repellent material.

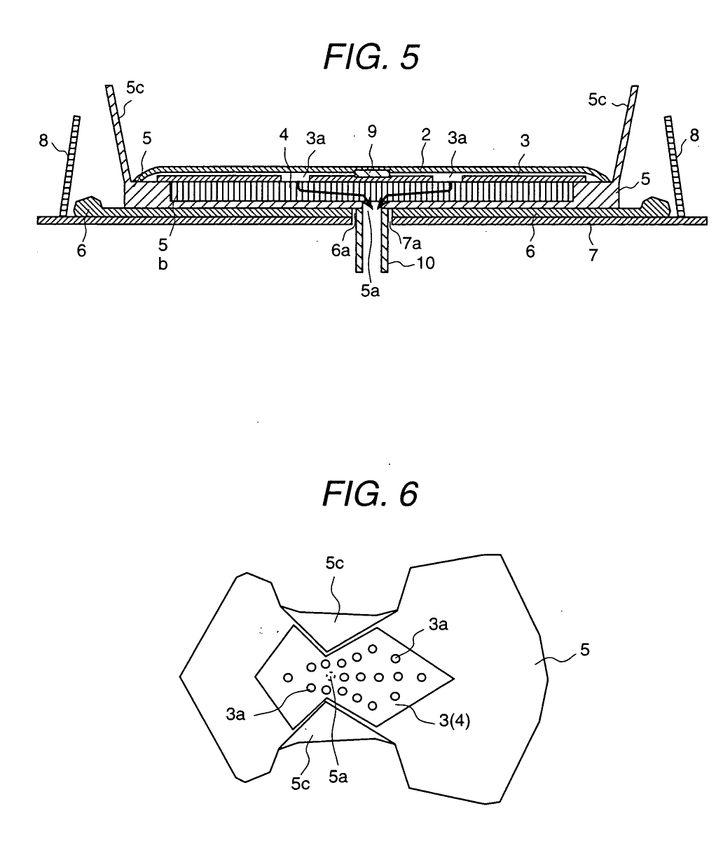

Along the periphery of the outer sheet 7 in the longitudinal direction (direction of the wearer's front and rear), three-dimensional gathers 8 are formed in such a way that they are slanted inwardly along the periphery of the outer sheet 7, as shown in FIG. 5. These slantingly provided three-dimensional gathers 8 prevent leaks from the sides caused by the wearer's physical activity or change of posture. In addition, a through hole 7a through which a urine drainage tube 10 passes is created in the outer sheet 7.

A urine absorbent sheet 6 adheres to the top surface of the outer sheet 7. The urine absorbent sheet 6 is provided to absorb urine which has not been collected by the support sheet 5, thereby preventing urine from wetting the wearer's clothes or bedding and also preventing urine from coming in contact with the wearer's skin. A through hole 6a, through which a urine drainage tube 10 passes, is formed in the urine absorbent sheet 6.

A support sheet 5 adheres to the surface of the urine absorbent sheet 6. The support sheet 5 has a concave portion or recess 5b into which the urine absorbing space forming sheet 4 can fit. Furthermore, a urine drainage port 5a, to which a urine drainage tube 10 is connected, is formed in the concave portion 5b. The urine drainage port 5a is located at a position which corresponds to an approximate center of the perforated urine absorbent sheet 3, which will be described later, on which a large number of urine drainage pores 3a are formed.

Both sides of the support sheet 5, which fit to the wearer's crotch (urinating part), are loose fitting, thereby forming peripheral pieces 5c, as shown in FIGS. 5 and 6, to prevent urine from leaking from the sides. As seen in FIG. 6, a urine absorbing space forming sheet 4 fits into the concave portion 5b, and a perforated urine absorbent sheet 3 is disposed on the top surface of the urine absorbing space forming sheet 4. The support sheet 5 is made of an elastic material, such as polyethylene foam.

The urine absorbing space forming sheet 4 is made of a porous, fibriform skeletal material with no water-absorption capability, and the sides of the sheet 4 adhere to the concave portion 5b of the support sheet 5, thereby molding into it. The urine absorbing space forming sheet 4 is made of a 5 to 10 mm thick, porous material to ensure that the air space for sucking out urine (urine collecting space) is maximized. The urine absorbing space forming sheet 4 fits into the concave portion 5b of the support sheet 5. This configuration eliminates a clearance or level difference between the urine absorbing space forming sheet 4 and the support sheet 5, thereby preventing urine from remaining.

A perforated urine absorbent sheet 3, on which a large number of urine drainage pores 3a are formed, is disposed on the top surface of the urine absorbing space forming sheet 4. The urine absorbing space forming sheet 4 is disposed such that it comes in close contact with the bottom surface of the perforated urine absorbent sheet 3. The perforated urine absorbent sheet 3 has a similar shape to that of the urine absorbing space forming sheet 4, and the bottom surface of its periphery adheres to the support sheet 5, as shown in FIG. 5. Thus, the urine absorbing space forming sheet 4 is disposed between the perforated urine absorbent sheet 3 and the support sheet 5.

Urine drainage pores 3a are formed in the perforated urine absorbent sheet 3 between the area where urine is discharged from the urinating part of the wearer and the vicinity of the buttocks in order to increase the percentage of urine collection, as well as to handle the spread of urine due to urination while the wearer is lying down on his/her back or is sitting. Urine drainage pores 3a are formed at locations with different distances necessary for effectively collecting urine. Furthermore, the diameter of the urine drainage pores 3a can vary according to the location from which urine is collected; for example, the diameter of the pore near the urinating part of the wearer can be made larger than that of other pores.

The top sheet 2 is disposed on the surface of the perforated urine absorbent sheet 3, and a urine sensor 9 is located between the top sheet 2 and the perforated urine absorbent sheet 3. The surface of the perforated urine absorbent sheet 3 is covered with the hard-breathable top sheet 2. Urine discharged by a wearer is absorbed by the top sheet 2 and then absorbed into the urine drainage pores 3a formed on the perforated urine absorbent sheet 3. The top sheet 2 is made of a liquid-permeable, hard-breathable nonwoven cloth which is made of, for example, polypropylene and polyolefin polyester blended with cotton so that friction between the wearer's skin and the fabric is minimized.

In addition, a mesh sheet makes up a part of the surface of the nonwoven cloth used as the top sheet 2 where it comes in contact with the wearer's urinating part and the surrounding skin. This is to increase the liquid-absorbent and sweat-absorbent capabilities so that urine can be quickly absorbed by the urine drainage pores 3a formed in the perforated urine absorbent sheet 3 through small pores created in the mesh sheet. Because urine can be quickly absorbed by the perforated urine absorbent sheet 3, the wearer has a minimal amount of discomfort due to moisture around the wearer's urinating part.

Herein, the hard breathability characteristic of the top sheet 2 will be explained. Hard breathability of the top sheet 2 means that the breathability measured according to the General Textile Testing Method's breathability testing method A, prescribed in JIS L1096, 6.27.1, is from 0 to 100 cc/cm²/second, and is preferably from 0 to 50 cc/cm²/second, when the top sheet 2 is moist. When the top sheet 2 is dry, the breathability is from 20 to 200 cc/cm²/second, preferably from 20 to 100 cc/cm²/second, and more preferably from 20 to 50 cc/cm²/second.

Moreover, “being moist” is a condition in which the moisture content (%) of the top sheet 2 that is obtained by the following Equation 1 is 100% or more, and “being dry” is a condition in which the top sheet 2 has been left dry in a 20° C. and RH60% atmosphere, or the condition of, what is called, official moisture regain.

Moisture content=(Weight of moist sheet−Weight of dry sheet)/(Weight of dry sheet) (Equation 1)

In FIG. 1, one end of the urine drainage tube (first urine drainage means) 10 is connected to the urine drainage port 5a formed on the support sheet 5. The urine drainage tube 10 penetrates holes 6a and 7a of the urine absorbent sheet 6 and the outer sheet 7, and the other end is connected to a one-touch joint 13. The one-touch joint 13 is mounted to one end of the urine drainage tube (second urine drainage means) 11. This joint 13 connects the other end of the urine drainage tube 10 to the end of the urine drainage tube 11. The urine drainage tubes 10 and 11 are made of soft, flexible materials such as soft resin, and the one-touch joint 13 is made of a soft material.

A urine tank 21 is sealed by a lid 22. The other end of the urine drainage tube 11 passes through the lid 22 of the urine tank 21 and is located in the vapor phase area 21a of the urine tank 21. One end of the vacuum tube 12 is connected to a vacuum pump 23, and the other end passes through the lid 22 of the urine tank 21 and is located in the vapor phase area 21a of the urine tank 21. Like the urine drainage tubes 10 and 11, the vacuum tube 12 is also made of a soft, flexible material. The capacity of the urine tank 21 is about 500 cm³, which is sufficient to store two separate urinations. This tank also comes in 200 cm³ or 1000 cm³, which allows for prolonged use at night.

The vacuum pump 23 is driven by a motor 24. The motor 24 uses a battery 25 as a driving power source, and it is controlled by a control device installed in the control board 26. The vacuum pump 23 is small, having a diameter of 30 mm×70 mm. The voltage of the battery 25 is approximately 6 V.

A urine sensor 9 detects when urine has been discharged in the top sheet 2. It is electrically conductive and detects the wearer's urination by sensing a resistance value change. The urine detection signal detected by the urine sensor 9 is inputted via signal lines 14 and 15 into the control board 26 that controls the vacuum pump 23. The signal lines 14 and 15 are connected by a one-touch joint 16.

In this configuration, the urine receptacle 1 is worn so that the top sheet 2 comes in contact with the urinating part of a wearer (not shown) in his/her underwear. The urine tank 21, vacuum pump 23, and the motor 24 can be carried by the wearer or can be placed on or under the bed on which the wearer lies.

When the wearer urinates in this situation, urine discharged in the urine receptacle 1 is absorbed by the top sheet (nonwoven cloth) 2. When the top sheet 2 absorbs urine, the space among fibers of the top sheet 2 is filled with urine. When the top sheet 2 absorbs urine, the urine sensor 9 is turned on, and a urine detection signal is inputted to the control board 26. The control device installed in the control board 26 activates the motor 24 to drive the vacuum pump 23.

When air in the urine tank 21 has been discharged by the vacuum pump 23, air pressure in the urine absorbing space inside the urine absorbing space forming sheet 4 decreases, creating negative pressure in a large number of urine drainage pores 3a formed on the perforated urine absorbent sheet 3. The top sheet 2 is hard-breathable and the outer sheet 7 is non-breathable. Therefore, pressure in the urine absorbing space of the urine absorbing space forming sheet 4 efficiently decreases.

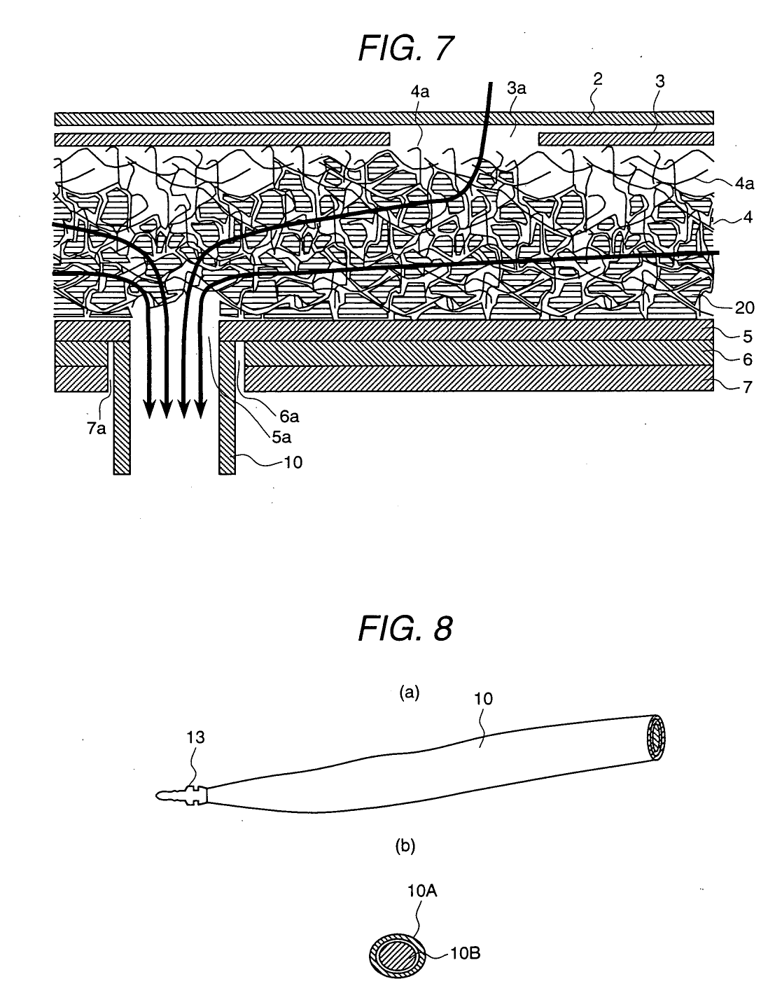

When pressure in the urine absorbing space of the urine absorbing space forming sheet 4 becomes negative, a uniform suction force is created in all of the urine drainage pores 3a. Consequently, it is possible to draw urine from a urine drainage pore 3a away from the urine drainage port 5a to the urine absorbing space. As shown in FIG. 7, urine 20 (indicated by horizontally hatched lines) flows into the pores of the fibrous material 4a which create a urine absorbing space 4A of the urine absorbing space forming sheet 4 and flows in the direction indicated by the thick arrows. Urine absorbed by the urine absorbing space forming sheet 4 is drawn into a urine drainage tube 10 by means of a suction force due to negative pressure.

The urine drainage pores 3a of the perforated urine absorbent sheet 3 are formed in such a way that, with the appropriate number of pores of a certain diameter, the urine drainage pores 3a properly maintain negative pressure in the urine absorbing space so as to draw urine into the urine absorbing space by means of a uniform suction force from a wide area of the top sheet 2 which comes in contact with the perforated urine absorbent sheet 3. That is, the perforated urine absorbent sheet 3 is provided in order to uniformly create negative pressure in the urine absorbing space of the urine absorbing space forming sheet 4. Without the perforated urine absorbent sheet 3, urine is exclusively drawn from the vicinity of the urine drainage port 5a (urine drainage tube 10) into the urine drainage tube 10, causing the percentage of urine collection to decrease.

Urine drawn into the urine drainage tube 10 is further drawn into the urine tank 21 by negative pressure via the urine drainage tube 11, and it is stored in the tank. The urine tank 21 can be removed by disconnecting the urine drainage tubes 10 and 11 by unlocking the one-touch joint 13 as well as by disconnecting the signal lines 14 and 15 by unlocking the one-touch joint 16. Thus, the automatic urine disposal device, including the urine tank 21, can be empited and urine stored therein can be disposed of. The urine tank 21, can be carried by removing the lid 22 from the urine tank 21 then and urine stored in the urine tank 21 can be disposed of.

Moreover, after the wearer has worn the urine receptacle 1 for a day or when it became dirty due to defecation, the used urine receptacle 1 is discarded by disconnecting the urine drainage tube 10 from the urine sensor 9 by unlocking the joints 13 and 16, and the wearer then engages a new urine receptacle 1.

A urine receptacle 1 according to the present invention is designed such that a urine absorbing space forming sheet 4 is disposed between a perforated urine absorbent sheet 3 and a support sheet 5, and urine is drained through a large number of urine drainage pores 3a formed in the perforated urine absorbent sheet 3 and is drawn into a sealed urine tank 21 by means of a vacuum pump 23 via urine drainage tubes 10 and 11. This configuration makes it possible to maintain a high percentage of urine collection even if the vacuum pump 23 has a low suction force.

FIG. 8 shows another embodiment of a urine drainage tube 10 which is connected to the urine receptacle 1. The urine drainage tube 10 shown in FIG. 8 is different from the urine drainage tube in the above-mentioned embodiment. In FIG. 8, a space-holding material 10B is provided on the inner surface of the outer shell 10A of the urine drainage tube 10. This configuration allows the outer shell 10A to be made of a soft material, such as liquid-impermeable vinyl, and to ensure a space for vacuuming urine without the tube being compressed by the wearer's weight. Furthermore, in the aforementioned embodiment, a soft flexible material, such as liquid-impermeable rubber or soft resin, must have sufficient rigidity so that it will not be compressed by a wearer's weight. However, the urine drainage tube 10 shown in FIG. 8 can have more flexibility, thereby reducing the discomfort to the wearer.

As state above, in the urine receptacle used in accordance with the present invention, urine discharged in a top sheet is absorbed through a large number of urine drainage pores formed on perforated urine absorbent sheet. Therefore, the percentage of urine collection increases and the amount of urine which remains in the urine receptacle is reduced. With the increase in the percentage of urine collection, it is possible for a small capacity vacuum pump with a low suction force to drain urine from the urine absorbent material. Therefore, it is possible to drain urine from the urine receptacle without discomfort to the wearer, and the device can be made compact and lightweight.

Since the device can be compact and lightweight, if it is used as a portable automatic urine disposal device, it will be most efficient. Furthermore, because the device is compact and lightweight and the vacuum pump does not unnecessarily absorb air, the noise created by the pump is minimal and urine can be quietly drained without bothering other patients in the room at night.

Furthermore, in the aforementioned embodiments, peripheral pieces provided on the support sheet prevent urine from leaking from the sides. Consequently, urine leaks from the sides can be doubly prevented by the peripheral pieces and the gathers formed on the outer sheet. However, it is obvious that the peripheral pieces need not be necessarily provided on the support sheet in the practical use of the urine receptacle.

Moreover, in the aforementioned embodiments, the top sheet is hard-breathable and the support sheet is non-breathable. However, if the pressure in the urine drainage pores of the perforated urine absorbent sheet is made negative, it is obvious that the same effect can be expected when those sheets are slightly breathable.

The present invention can increase the percentage of urine collection by the urine receptacle and reduce the amount of urine which remains in the urine receptacle. Therefore, it is possible for a small capacity vacuum pump to drain urine from the urine receptacle. As a result, it is possible to drain urine from the urine receptacle without discomfort to the wearer, and the device can be compact and lightweight.

Since the device can be compact and lightweight, if it is used as a portable automatic urine disposal device, it will be most efficient. Furthermore, because the device is compact and lightweight and the vacuum pump does not unnecessarily absorb air, the noise created by the pump is minimal and urine can be quietly drained without bothering other patients in the room at night.

1. An automatic urine disposal device for absorbing urine collected in a urine receptacle by means of a vacuum pump and directing it to a urine tank, said urine receptacle comprising
a top sheet which forms said urine receptacle's surface which comes in contact with the wearer's skin,
a perforated urine absorbent sheet disposed adjacent said top sheet of on which a large number of urine drainage pores for absorbing urine discharged in said top sheet are formed,
a support sheet disposed on said top sheet's opposite side which does not come in contact with the wearer's skin, and
a urine absorbing space forming sheet which is disposed closer to the support sheet than said perforated urine absorbent sheet, characterized in that
said urine tank is sealed,
said support sheet has a first urine drainage means,
a second urine drainage means for directing urine into said urine tank is connected to said first urine drainage means, and
said vacuum pump creates a vacuum in the inside of said urine tank.

2. An automatic urine disposal device according to claim 1, characterized in that said second urine drainage means is a tube, and said vacuum pump sucks out air from said urine tank.

3. An automatic urine disposal device according to claim 1, characterized in that
said urine receptacle has a urine sensor for detecting that urine has been discharged in said urine receptacle and for activating said vacuum pump,
said support sheet is liquid-impermeable and non-breathable, and
said vacuum pump decreases the pressure in said urine tank and draws urine from a urine drainage means formed on said support sheet to said urine tank.

4. An automatic urine disposal device according to claim 3, characterized in that
said urine tank has a lid for sealing said urine tank,
said second urine drainage means directs urine from a first urine drainage means formed on said support sheet to said urine tank via said lid,
said vacuum pump makes the pressure of the urine drainage pores of said perforated urine absorbent sheet negative and absorbs sucks out urine into said urine tank.

5. An automatic urine disposal device according to claim 3, characterized in that
said top sheet is liquid-permeable and hard-breathable,
said urine absorbing space forming sheet is fibrous, and
said first urine drainage means is connected to said second urine drainage means by a joint.

6. An automatic urine disposal device according to claim 1, characterized in that
said top sheet covers the surface of said perforated urine absorbent sheet,
said urine absorbing space forming sheet is disposed between the bottom surface of said perforated urine absorbent sheet and said support sheet.

7. An automatic urine disposal device according to claim 6, characterized in that
said top sheet is hard-breathable, and
said support sheet is liquid-impermeable.

8. An automatic urine disposal device according to claim 1, characterized in that
said top sheet is liquid-permeable and hard-breathable and covers the surface of said perforated urine absorbent sheet,
said urine absorbing space forming sheet is disposed such that it comes in close contact with the bottom surface of said perforated urine absorbent sheet thereby forming a urine absorbing space,
said support sheet is liquid-impermeable and supports said urine absorbing space forming sheet, and
said urine receptacle has an outer sheet which holds the aforementioned sheets.

9. A urine receptacle for use in an automatic urine disposal device to absorb urine discharged from a wearer's urinating part, said urine receptacle comprising
a perforated urine absorbent sheet for absorbing discharged urine,
a top sheet which covers the surface of said perforated urine absorbent sheet,
a urine absorbing space forming sheet which is disposed such that it comes in close contact with the bottom surface of said perforated urine absorbent sheet, and
a liquid-impermeable support sheet which supports said urine absorbing space forming sheet and has a urine drainage means.

10. A urine receptacle according to claim 9, characterized in that
said perforated urine absorbent sheet and said support sheet are liquid-impermeable, and
said top sheet is hard-breathable.

11. A urine receptacle according to claim 9, characterized in that
said perforated urine absorbent sheet and said support sheet are liquid-impermeable and non-breathable, and
said top sheet is liquid-permeable and hard-breathable.

12. A urine receptacle according to claim 11, characterized in that
said urine absorbing space forming sheet is made of a fibrous material, and
an outer sheet is provided to support said top sheet, said perforated urine absorbent sheet, said urine absorbing space forming sheet and said support sheet.

13. A urine receptacle according to claim 12, characterized in that
gathers are provided along the periphery of said support sheet.

14. A urine receptacle according to claim 13, characterized in that
said urine absorbing space forming sheet fits into the concave portion of said support sheet,
said outer sheet contains said top sheet, said perforated urine absorbent sheet, said urine absorbing space forming sheet and said support sheet, and
said urine absorbent sheet is disposed between said support sheet and said outer sheet.
